(19) United States
(12) Patent Application Publication
(10) Pub. No.: US 2022/0071889 A1
(43) Pub. Date: Mar. 10, 2022

(54) ANHYDROUS COSMETIC COMPOSITION COMPRISING A LIQUID FATTY SUBSTANCE, A SOLID FATTY SUBSTANCE AND A SURFACTANT

(71) Applicant: L'Oreal, Paris (FR)

(72) Inventors: Anne-Laure Fameau, SAINT OUEN (FR); Koudedji Sow-Kebe, SAINT OUEN (FR)

(73) Assignee: L'Oreal, Paris (FR)

(21) Appl. No.: 17/416,245

(22) PCT Filed: Dec. 19, 2019
(86) PCT No.: PCT/EP2019/086354

§ 371 Date: Jun. 18, 2021

(30) Foreign Application Priority Data
Dec. 20, 2018 (FR) 1873704

(51) Int. Cl. A61K 8/92; A61Q 19/10; A61Q 19/00; A61Q 5/02; A61Q 5/12; A61K 8/34; A61K 8/37; A61K 8/04

(52) U.S. Cl. CPC A61K 8/922; A61Q 19/10; A61Q 19/007; A61Q 5/02; A61K 2800/31; A61K 8/342; A61K 8/375; A61K 8/046; A61Q 5/12

(57) ABSTRACT

The invention relates to an anhydrous cosmetic composition in foam form comprising, at 25° C. and atmospheric pressure, at least one solid fatty substance, at least one liquid fatty substance and at least one particular surfactant. The invention also relates to a cosmetic process for treating keratin materials, in particular human keratin materials such as the hair and the skin, comprising the application of said anhydrous cosmetic composition. The present invention also relates to the use of said cosmetic composition for washing and/or conditioning keratin materials, in particular human keratin materials such as the hair or the skin.

The invention relates to an anhydrous cosmetic composition comprising, at 25° C. and atmospheric pressure, at least one solid fatty substance, at least one liquid fatty substance and at least one particular surfactant.

The invention also relates to a cosmetic process for treating keratin materials, in particular human keratin materials such as the hair and the skin, comprising the application of said anhydrous cosmetic composition.

The present invention also relates to the use of said cosmetic composition for washing and/or conditioning keratin materials, in particular human keratin materials such as the hair or the skin.

The cleansing of keratin materials, notably such as the hair and the skin, is very important. The reason for this is that it allows the removal of the diverse soiling initially present on the hair or the skin, such as excess sebum or residues of cosmetic products or of makeup used daily.

To do this, it is known practice to use foaming detergent aqueous gels. Their cleansing action is provided by the surfactants they contain, these surfactants placing in suspension the fatty residues and the pigments of the makeup products. These gels are efficient and pleasant to use since they give a good foam.

In the field of washing and/or conditioning keratin materials, notably human keratin materials and better still the skin and the hair, use may be made of shower gels and conditioning shampoos. The latter products are generally formulated on the basis of micellar surfactant aggregates.

During the use of a foaming product, whether for the hair or the skin, it is sought to have a product which gives good foam qualities, i.e. a foam which forms quickly and easily, in an abundant amount, having a firm texture and good persistence (not running), and which rinses off rapidly.

However, it was found that the production of a foam of sufficient quality and in sufficient quantity, which keeps these properties, notably in terms of quality and quantity over time, could be improved.

The existing compositions also have the drawback of requiring a lot of rinsing water in order to remove the surplus product from the keratin materials. In many countries where access to water is restricted, the rinsing time and therefore the amount of water required to properly rinse off the product are key indicators of the use qualities of a composition.

Thus, there is a real need for a cosmetic composition for washing and/or conditioning keratin materials, in particular human keratin materials such as the hair or the skin, which does not have the drawbacks mentioned above, i.e. a composition which has good foam properties and which is easy to apply to keratin materials. The composition must also rinse off easily, while at the same time giving the keratin materials good cosmetic and sensory properties, notably in terms of softness, without making them feel greasy.

It has been discovered, surprisingly, that a combination of at least one particular surfactant with an anhydrous composition composed of fatty substance having a melting point of greater than 25° C. and of fatty substance having a melting point of less than or equal to 25° C. makes it possible to achieve the objectives presented above. Specifically, this particular combination makes it possible to obtain a foam which has a firm and good-quality texture, which is stable over time. The composition also has good use qualities, notably as regards the ease of application and of spreading on keratin materials, and also good rinseability.

This composition also gives keratin materials good cosmetic and sensory properties.

One subject of the invention is thus an anhydrous cosmetic composition comprising:
one or more fatty substances with a melting point of less than or equal to 25° C.,
one or more fatty substances with a melting point of greater than 25° C., and
one or more surfactants with a melting point of less than or equal to 25° C., said composition being in the form of a foam.

The composition according to the invention makes is under the form of an abundant foam, which has a good stability over time.

Furthermore, the foam composition according to the invention has a firm and creamy texture, and allows easy spreading and distribution of the product on the keratin materials, and notably the hair or the skin, while at the same time having good rinseability and a very favorable water footprint.

The composition of the invention also gives keratin materials good cosmetic properties, notably in terms of feel and softness. As regards keratin materials such as the hair, they in particular give the hair a natural, non-greasy and non-laden feel, making the hair smooth and soft. Thus, keratin materials treated with the composition of the invention have a pleasant sensation of softness, lightness and suppleness.

A subject of the invention is also a cosmetic process for treating keratin materials, in particular human keratin materials such as the hair and the skin, comprising the application to said keratin materials of an anhydrous composition as defined previously.

A subject of the invention is also the use of the anhydrous cosmetic composition as defined previously for washing and/or conditioning keratin materials, notably human keratin materials such as the hair and the skin.

Other subjects, features, aspects and advantages of the invention will emerge even more clearly on reading the description and the examples that follow.

For the purposes of the present invention, the cosmetic composition is anhydrous, i.e. said composition comprises a water content of less than or equal to 2% by weight, preferably less than or equal to 1% by weight, more preferentially less than or equal to 0.5% by weight, even more preferentially less than or equal to 0.1% by weight, relative to the total weight of the composition. Preferably, the composition according to the invention is free of water (0%).

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name MDSC 2920 by the company TA Instruments. In the present patent application, all the melting points are determined at atmospheric pressure (1.013×10⁵ Pa).

The term “fatty substance” means an organic compound that is insoluble in water at 25° C. and at atmospheric pressure (1.013×10⁵ Pa) (solubility of less than 5% by weight, preferably less than 1% by weight and even more preferentially less than 0.1% by weight). They bear in their structure at least one hydrocarbon-based chain including at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

Liquid Fatty Substances

The anhydrous cosmetic composition according to the present invention comprises one or more fatty substances with a melting point of less than or equal to 25° C., preferably less than or equal to 20° C. at atmospheric pressure (1.013×10⁵ Pa). In other words, these fatty substances are liquid at atmospheric pressure, and are not in a solid or gaseous state. In the present patent application, this or these fatty substances are also referred to as “liquid fatty substances” or “oils”.

Advantageously, the liquid fatty substances that may be used in the present invention are neither (poly)oxyalkylenated nor (poly)glycerolated.

The term “non-silicone fatty substance” refers to a fatty substance not containing any Si—O bonds and the term “silicone fatty substance” refers to a fatty substance containing at least one Si—O bond.

Preferably, the liquid fatty substances are non-silicone substances.

More particularly, the fatty substance(s) with a melting point of less than or equal to 25° C. according to the invention are chosen from C₆ to C₁₆ liquid hydrocarbons, liquid hydrocarbons comprising more than 16 carbon atoms, non-silicone oils of animal origin, oils of triglyceride type of plant or synthetic origin, fluoro oils, liquid fatty alcohols, liquid fatty acid and/or fatty alcohol esters other than triglycerides, and silicone oils, and mixtures thereof.

It is recalled that the fatty alcohols, esters and acids more particularly contain at least one saturated or unsaturated, linear or branched hydrocarbon-based group, comprising from 6 to 40 and better still from 8 to 30 carbon atoms, which is optionally substituted, in particular, with one or more hydroxyl groups (in particular 1 to 4). If they are unsaturated, these compounds may comprise one to three conjugated or unconjugated carbon-carbon double bonds.

As regards the liquid C₆-C₁₆ hydrocarbons, they may be linear, branched, and optionally cyclic, and are preferably chosen from alkanes. Examples that may be mentioned include hexane, cyclohexane, undecane, dodecane, isododecane, tridecane or isoparaffins, such as isohexadecane or isodecane, and mixtures thereof.

The liquid hydrocarbons comprising more than 16 carbon atoms may be linear or branched, of mineral or synthetic origin, and are preferably chosen from liquid paraffin or liquid petroleum jelly, polydecenes, hydrogenated polyisobutene such as Parleam©, and mixtures thereof.

A hydrocarbon-based oil of animal origin that may be mentioned is perhydrosqualene.

The triglyceride oils of plant or synthetic origin are preferably chosen from liquid fatty acid triglycerides including from 6 to 30 carbon atoms, for instance heptanoic or octanoic acid triglycerides, or alternatively, for example, sunflower oil, corn oil, soybean oil, pumpkin oil, grapeseed oil, sesame seed oil, hazelnut oil, apricot oil, macadamia oil, arara oil, castor oil, avocado oil, caprylic/capric acid triglycerides, for instance those sold by the company Stearinerie Dubois or those sold under the names Miglyol© 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec© PC1 and Flutec© PC3 by the company BNFL Fluorochemicals; perfluoro-1,2-dimethylcyclobutane; perfluoroalkanes such as dodecafluoropentane and tetradecafluorohexane, sold under the names PF 5050© and PF 5060© by the company 3M, or bromoperfluorooctyl sold under the name Foralkyl© by the company Atochem; nonafluoromethoxybutane and nonafluoroethoxyisobutane; perfluoromorpholine derivatives such as 4-trifluoromethyl perfluoromorpholine sold under the name PF 5052© by the company 3M.

The liquid fatty alcohols that are suitable for use in the invention are more particularly chosen from linear or branched, saturated or unsaturated alcohols, preferably unsaturated or branched alcohols, including from 6 to 40 carbon atoms and preferably from 8 to 30 carbon atoms. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof.

As regards the liquid esters of fatty acids and/or of fatty alcohols other than the triglycerides mentioned above, mention may be made notably of esters of saturated or unsaturated, linear C₁ to C₂₆ or branched C₃ to C₂₆ aliphatic monoacids or polyacids and of saturated or unsaturated, linear C₁ to C₂₆ or branched C₃ to C₂₆ aliphatic monoalcohols or polyalcohols, the total carbon number of the esters being greater than or equal to 6 and more advantageously greater than or equal to 10.

Preferably, for the esters of monoalcohols, at least one from among the alcohol and the acid from which the esters of the invention are derived is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononate; octyldodecyl erucate; oleyl erucate; ethyl palmitate, isopropyl palmitate, such as 2-ethylhexyl palmitate, 2-octyldecyl palmitate; alkyl myristates such as isopropyl 2-octyldodecyl myristate, isobutyl stearate; 2-hexyldecyl laurate, and mixtures thereof.

Preferably, among the monoesters of monoacids and of monoalcohols, use will be made of ethyl palmitate, isopropyl palmitate, alkyl myristates such as isopropyl myristate or ethyl myristate, isocetyl stearate, 2-ethylhexyl isononanoate, isodecyl neopentanoate and isostearyl neopentanoate, and mixtures thereof.

Still within the context of this variant, esters of C₄ to C₂₂ dicarboxylic or tricarboxylic acids and of C₁ to C₂₂ alcohols and esters of mono-, di- or tricarboxylic acids and of C₂ to C₂₆ di-, tri-, tetra- or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C₆ to C₃₀ and preferably C₁₂ to C₂₂ fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be chosen notably from the group comprising the esters or mixtures of esters of sugars described previously and of linear or branched, saturated or unsaturated C₆ to C₃₀ and preferably C₁₂ to C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates, arachidonates or mixtures thereof such as, in particular, the mixed esters oleo-palmitate, oleo-stearate and palmito-stearate.

More particularly, use is made of monoesters and diesters and in particular sucrose, glucose or methylglucose mono- or dioleates, stearates, behenates, oleopalmitates, linoleates, linolenates and oleostearates, and mixtures thereof.

Mention may be made, by way of example, of the product sold under the name Glucate© DO by the company Amerchol, which is a methylglucose dioleate.

Preferably, use will be made of a liquid ester of a monoacid and of a monoalcohol.

The silicone oils that may be used in the composition according to the present invention may be volatile or non-volatile, cyclic, linear or branched silicone oils, which are unmodified or modified with organic groups, and preferably have a viscosity from 5×10⁻⁶ to 2.5 m²/s at 25° C., and preferably 1×10⁻⁵ to 1 m²/s.

Preferably, the silicone oils are chosen from polydialkylsiloxanes, notably polydimethylsiloxanes (PDMS), and liquid polyorganosiloxanes including at least one aryl group.

These silicone oils may also be organomodified. The organomodified silicone oils that may be used in accordance with the invention are preferably liquid silicones as defined above and including in their structure one or more organofunctional groups attached via a hydrocarbon-based group, chosen, for example, from amine groups and alkoxy groups.

Organopolysiloxanes are defined in greater detail in Walter Noll's Chemistry and Technology of Silicones (1968), Academic Press. They may be volatile or non-volatile.

When they are volatile, the silicone oils are more particularly chosen from those with a boiling point of between 60° C. and 260° C., and even more particularly from:

(i) cyclic polydialkylsiloxanes including from 3 to 7 and preferably from 4 to 5 silicon atoms. These are, for example, octamethylcyclotetrasiloxane sold notably under the name Volatile Silicone© 7207 by Union Carbide or Silbione© 70045 V2 by Rhodia, decamethylcyclopentasiloxane sold under the name Volatile Silicone© 7158 by Union Carbide, and Silbione© 70045 V5 by Rhodia, and mixtures thereof.

Mention may also be made of cyclocopolymers of the dimethylsiloxane/methylalkylsiloxane type, such as Volatile Silicone© FZ 3109 sold by the company Union Carbide.

Mention may also be made of mixtures of cyclic polydialkylsiloxanes with organosilicon compounds, such as the mixture of octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pages 27-32—Todd & Byers Volatile Silicone Fluids for Cosmetics.

Non-volatile polydialkylsiloxanes are preferably used.

These silicone oils are more particularly chosen from polydialkylsiloxanes, among which mention may be made mainly of polydimethylsiloxanes bearing trimethylsilyl end groups. The viscosity of the silicones is measured at 25° C. according to ASTM standard 445 Appendix C.

Among these polydialkylsiloxanes, mention may be made, in a non-limiting manner, of the following commercial products:
the Silbione© oils of the 47 and 70 047 series or the Mirasil© oils sold by Rhodia, for instance the oil 70 047 V 500 000;
the oils of the Mirasil© series sold by the company Rhodia;
the oils of the 200 series from the company Dow Corning, such as DC200 with a viscosity of 60 000 mm²/s;
the Viscasil© oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from the company Rhodia.

The organomodified silicones that may be used in accordance with the invention are silicones as defined above and including in their structure one or more organofunctional groups attached via a hydrocarbon-based group.

As regards the liquid polyorganosiloxanes including at least one aryl group, they may notably be polydiphenylsiloxanes, and polyalkylarylsiloxanes functionalized with the organofunctional groups mentioned previously.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, examples that may be mentioned include the products sold under the following names:
the Silbione® oils of the 70 641 series from Rhodia;
the oils of the Rhodorsil© 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Among the organomodified silicones, mention may be made of polyorganosiloxanes including:
substituted or unsubstituted amino groups, such as the products sold under the names GP 4 Silicone Fluid and GP 7100 by the company Genesee or the products sold under the names Q2 8220 and Dow Corning 929 or 939 by the company Dow Corning. The substituted amino groups are in particular C1 to C4 aminoalkyl groups;
alkoxy groups,
hydroxyl groups.

Preferably, the fatty substance(s) with a melting point of less than or equal to 25° C. are chosen from triglyceride oils of plant origin, and mixtures thereof, and more preferentially from sunflower oil, soybean oil, olive oil, avocado oil, and mixtures thereof.

The total content of the fatty substance(s) with a melting point of less than or equal to 25° C. present in the composition according to the invention is advantageously greater than or equal to 70% by weight, preferably greater than or equal to 75% by weight, more preferentially from 75% to 95% by weight and better still from 80% to 90% by weight, relative to the total weight of the composition.

Preferably, the composition does not comprise any silicone liquid fatty substances.

Preferably, the total content of the non-silicone fatty substance(s) with a melting point of less than or equal to 25° C. present in the composition according to the invention is advantageously greater than or equal to 70% by weight, preferably greater than or equal to 75% by weight, more preferentially from 75% to 95% by weight and better still from 80% to 90% by weight, relative to the total weight of the composition.

Solid Fatty Substances

The anhydrous cosmetic composition according to the present invention also comprises one or more fatty substances with a melting point of greater than 25° C., preferably greater than or equal to 28° C., more preferentially greater than or equal to 30° C. at atmospheric pressure (1.013×10⁵ Pa). In the present patent application, this or these fatty substances are also referred to as “solid fatty substance(s)”.

Advantageously, the solid fatty substances that may be used in the present invention are neither (poly)oxyalkylenated nor (poly)glycerolated.

The solid fatty substances according to the invention preferably have a viscosity of greater than 2 Pa·s, measured at 25° C. and at a shear rate of 1 s.

Preferably, the solid fatty substances are non-silicone substances.

The fatty substance(s) with a melting point of greater than 25° C. are preferably chosen from solid fatty acids, solid fatty alcohols, solid esters of fatty acids and/or of fatty alcohols, waxes, ceramides, monoglycerides, diglycerides or triglycerides, and mixtures thereof.

The term “fatty acid” means a long-chain carboxylic acid comprising from 6 to 40 carbon atoms, preferably from 8 to 30 carbon atoms. The solid fatty acids according to the invention preferentially comprise from 10 to 30 carbon atoms and better still from 14 to 22 carbon atoms. These fatty acids are neither oxyalkylenated nor glycerolated.

The solid fatty acids that may be used in the present invention are notably chosen from myristic acid, cetylic acid, stearylic acid, palmitic acid, arachidic acid, stearic acid, lauric acid, behenic acid, and mixtures thereof.

Particularly preferably, the fatty acid(s) are chosen from behenic acid and arachidic acid.

The term “fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated.

The solid fatty alcohols may be saturated or unsaturated, and linear or branched, and include from 8 to 40 carbon atoms, preferably from 10 to 30 carbon atoms. Preferably, the solid fatty alcohols have the structure R—OH with R denoting a linear alkyl group, optionally substituted with one or more hydroxyl groups, comprising from 8 to 40, preferentially from 10 to 30 carbon atoms, better still from 10 to 30, or even from 12 to 24 atoms and even better still from 14 to 22 carbon atoms.

The solid fatty alcohols that may be used are preferably chosen from saturated or unsaturated, linear or branched, preferably linear and saturated, (mono)alcohols including from 8 to 40 carbon atoms, better still from 10 to 30, or even from 12 to 24 atoms and better still from 14 to 22 carbon atoms.

The solid fatty alcohols that may be used may be chosen, alone or as a mixture, from:
myristyl alcohol (or 1-tetradecanol);
cetyl alcohol (or 1-hexadecanol);
stearyl alcohol (or 1-octadecanol);
arachidyl alcohol (or 1-eicosanol);
behenyl alcohol (or 1-docosanol);
lignoceryl alcohol (or 1-tetracosanol);
ceryl alcohol (or 1-hexacosanol);
montanyl alcohol (or 1-octacosanol);
myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl alcohol or cetearyl alcohol. Particularly preferably, the solid fatty alcohol is behenyl alcohol.

The solid esters of a fatty acid and/or of a fatty alcohol that may be used are preferably chosen from esters derived from a C₉-C₂₆ carboxylic fatty acid and/or from a C₉-C₂₆ fatty alcohol.

Preferably, these solid fatty esters are esters of a linear or branched, saturated carboxylic acid comprising at least 10 carbon atoms, preferably from 10 to 30 carbon atoms and more particularly from 12 to 24 carbon atoms, and of a linear or branched, saturated monoalcohol, comprising at least 10 carbon atoms, preferably from 10 to 30 carbon atoms and more particularly from 12 to 24 carbon atoms. The saturated carboxylic acids may be optionally hydroxylated, and are preferably monocarboxylic acids.

Esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may in particular be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C₉-C₂₆ alkyl palmitates, notably myristyl, cetyl or stearyl palmitate; C₉-C₂₆ alkyl myristates, such as cetyl myristate, stearyl myristate and myristyl myristate; and C₉-C₂₆ alkyl stearates, in particular myristyl stearate, cetyl stearate and stearyl stearate; and mixtures thereof.

For the purposes of the present invention, a wax is a lipophilic compound, which is solid at 25° C. and atmospheric pressure, with a reversible solid/liquid change of state, having a melting point greater than about 40° C., which may be up to 200° C., and having in the solid state anisotropic crystal organization. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to room temperature, recrystallization of the wax, which is microscopically and macroscopically detectable (opalescence), is obtained.

In particular, the waxes that are suitable for use in the invention may be chosen from waxes of animal, plant or mineral origin, non-silicone synthetic waxes, and mixtures thereof.

Mention may be made notably of hydrocarbon-based waxes, for instance beeswax, notably of biological origin, lanolin wax and Chinese insect waxes; rice bran wax, carnauba wax, candelilla wax, ouricury wax, alfalfa wax, berry wax, shellac wax, Japan wax and sumach wax; montan wax, orange wax, lemon wax, microcrystalline waxes, paraffins and ozokerite; polyethylene waxes, the waxes obtained by Fischer-Tropsch synthesis and waxy copolymers, and also esters thereof.

Mention may also be made of C₂ to C₆₀ microcrystalline waxes, such as Microwax HW.

Mention may also be made of the PM 500 polyethylene wax sold under the reference Permalen 50-L polyethylene.

Mention may also be made of waxes obtained by catalytic hydrogenation of animal or plant oils containing linear or branched C₈ to C₃₂ fatty chains. Among these waxes mention may notably be made of isomerized jojoba oil such as the trans-isomerized partially hydrogenated jojoba oil, notably the product manufactured or sold by the company Desert Whale under the commercial reference Iso-Jojoba-50@, hydrogenated sunflower oil, hydrogenated castor oil, hydrogenated coconut oil, hydrogenated lanolin oil and bis(1,1,1-trimethylolpropane) tetrastearate, notably the product sold under the name Hest 2T-4S© by the company Heterene.

The waxes obtained by hydrogenation of castor oil esterified with cetyl alcohol, such as those sold under the names Phytowax ricin 16L64@ and 22L73@ by the company Sophim, may also be used.

A wax that may be also used is a C₂₀ to C₄₀ alkyl (hydroxystearyloxy)stearate (the alkyl group comprising from 20 to 40 carbon atoms), alone or as a mixture. Such a wax is notably sold under the names Kester Wax K 82 P®, Hydroxypolyester K 82 P® and Kester Wax K 80 P® by the company Koster Keunen.

It is also possible to use microwaxes in the compositions of the invention; mention may be made notably of carnauba microwaxes, such as the product sold under the name MicroCare 350© by the company Micro Powders, synthetic-wax microwaxes, such as the product sold under the name MicroEase 114S© by the company Micro Powders, microwaxes consisting of a mixture of carnauba wax and polyethylene wax, such as the products sold under the names Micro Care 300© and 310© by the company Micro Powders, microwaxes consisting of a mixture of carnauba wax and of synthetic wax, such as the product sold under the name Micro Care 325© by the company Micro Powders, polyethylene microwaxes, such as the products sold under the names Micropoly 200©, 220©, 220L© and 250S© by the company Micro Powders, and polytetrafluoroethylene microwaxes, such as the products sold under the names Microslip 519© and 519 L© by the company Micro Powders.

The waxes are preferably chosen from mineral waxes, for instance paraffin, petroleum jelly, lignite or ozokerite wax; plant waxes, for instance cocoa butter or cork fibre or sugar cane waxes, olive tree wax, rice wax, hydrogenated jojoba wax, ouricury wax, carnauba wax, candelilla wax, alfalfa wax, or absolute waxes of flowers, such as essential wax of blackcurrant flower sold by the company Bertin (France); waxes of animal origin, for instance beeswaxes or modified beeswaxes (cerabellina), spermaceti, lanolin wax and lanolin derivatives; microcrystalline waxes; and mixtures thereof.

The ceramides, or ceramide analogues such as glycoceramides, that may be used in the compositions according to the invention, are known; mention may in particular be made of ceramides of classes I, II, III and V according to the Dawning classification.

The ceramides or analogues thereof that may be used preferably correspond to the following formula:

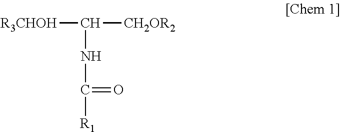

in which:
R₁ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R₂ denotes a hydrogen atom, a (glycosyl)ₙ group, a (galactosyl)ₘ group or a sulfogalactosyl group, in which n is an integer ranging from 1 to 4 and m is an integer ranging from 1 to 8;
R₃ denotes a C₁₅-C₂₆ hydrocarbon-based group, which is saturated or unsaturated in the alpha position, this group possibly being substituted with one or more C₁-C₁₄ alkyl groups;
it being understood that, in the case of natural ceramides or glycoceramides, R₃ may also denote a C₁₅-C₂₆ alpha-hydroxyalkyl group, the hydroxyl group optionally being esterified with a C₁₆-C₃₀ alpha-hydroxy acid.

The ceramides that are more particularly preferred are the compounds for which R₁ denotes a saturated or unsaturated alkyl derived from C₁₆-C₂₂ fatty acids; R₂ denotes a hydrogen atom and R₃ denotes a saturated linear C₁₅ group.

Preferentially, use is made of ceramides for which R₁ denotes a saturated or unsaturated alkyl group derived from C₁₄-C₃₀ fatty acids; R₂ denotes a galactosyl or sulfogalactosyl group; and R₃ denotes a —CH═CH—(CH₂)₁₂—CH₃ group.

Use may also be made of the compounds for which R₁ denotes a saturated or unsaturated alkyl radical derived from C₁₂-C₂₂ fatty acids; R₂ denotes a galactosyl or sulfogalactosyl radical; and R₃ denotes a saturated or unsaturated C₁₂-C₂₂ hydrocarbon-based radical and preferably a —CH═CH—(CH₂)₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4 triol and in particular N-stearoylphytosphingosine; 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof. N-oleoyldihydrosphingosine will preferably be used.

Preferably, the fatty substance(s) with a melting point of greater than 25° C. are chosen from solid fatty acids, solid fatty alcohols and mixtures thereof, and more preferentially from behenyl alcohol, behenic acid, and mixtures thereof.

The total content of the fatty substance(s) with a melting point of greater than 25° C. advantageously ranges from 1% to 20% by weight, preferably from 5% to 15% by weight, relative to the total weight of the composition.

Preferably, the total content of the non-silicone fatty substance(s) with a melting point of greater than 25° C. advantageously ranges from 1% to 20% by weight, preferably from 5% to 15% by weight, relative to the total weight of the composition.

The weight ratio between the total content of the fatty substance(s) with a melting point of less than or equal to 25° C. and the total content of the fatty substance(s) with a melting point of greater than 25° C. is advantageously greater than or equal to 3.5, preferably greater than 5.

Preferably, total content of the fatty substance(s) (i.e. of the fatty substance(s) with a melting point of less than or equal to 25° C. and of the fatty substance(s) with a melting point of greater than 25° C.) is greater than or equal to 75% by weight, preferably greater than or equal to 80% by weight, more preferentially greater than or equal to 85% by weight, relative to the total weight of the composition.

The weight ratio between the total content of the non-silicone fatty substance(s) with a melting point of less than or equal to 25° C. and the total content of the non-silicone fatty substance(s) with a melting point of greater than 25° C. is advantageously greater than or equal to 3.5, preferably greater than 5.

Preferably, the total content of the non-silicone fatty substance(s) (i.e. of the fatty substance(s) with a melting point of less than or equal to 25° C. and of the non-silicone fatty substance(s) with a melting point of greater than 25° C.) is greater than or equal to 75% by weight, preferably greater than or equal to 80% by weight, preferentially greater than or equal to 85% by weight, relative to the total weight of the composition.

Liquid Surfactants

The anhydrous cosmetic composition according to the present invention also comprises one or more surfactants with a melting point of less than or equal to 25° C. In the present patent application, this or these surfactants are also referred to as “liquid surfactant(s)”. The liquid surfactants according to the invention may be at 25° C. in a liquid form from very fluid to pasty. The liquid surfactants according to the invention are different from the liquid fatty substances described previously.

The surfactant(s) with a melting point of less than or equal to 25° C. may be chosen from anionic liquid surfactants, nonionic liquid surfactants, cationic liquid surfactants, amphoteric or zwitterionic liquid surfactants, and mixtures thereof.

Preferably, the surfactant(s) with a melting point of less than or equal to 25° C. are chosen from nonionic liquid surfactants, and mixtures thereof.

Preferably, the surfactant(s) with a melting point of less than or equal to 25° C. have an HLB value ranging from 1 to 9 and preferably from 1 to 7.

The term HLB is well known to those skilled in the art, and denotes the hydrophilic-lipophilic balance of a surfactant at 25° C. in the Griffin sense.

The term “hydrophilic-lipophilic balance (HLB)” means the equilibrium between the size and the strength of the hydrophilic group and the size and the strength of the lipophilic group of the surfactant. The HLB value according to Griffin is defined in J. Soc. Cosm. Chem. 1954 (volume 5), pages 249-256.

The surfactant(s) with a melting point of less than or equal to 25° C. are preferably chosen from liquid surfactants with an HLB value ranging from 1 to 9 and advantageously from liquid surfactants of the following families:

a) esters of (poly)glycerol comprising from 1 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s); these compounds may be chosen from:
monoesters of glycerol and of linear or branched, saturated or unsaturated, preferably branched or unsaturated, better still unsaturated, C₆-C₃₀, preferably C₈-C₂₄, better still C₁₀-C₂₀ fatty acid(s),
monoesters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s); examples that may be mentioned include diglyceryl monooleate (INCI name: polyglyceryl-2 oleate), for instance the product sold by Taiyo Kagaku under the name Sunsoft Q-17B, polyglyceryl-2 laurate, for instance the product sold by Taiyo Kagaku under the name Sunsoft Q-12D;
diesters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s);
triesters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s);
pentaesters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s);
hexaesters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s);
heptaesters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s);
decaesters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s);
polyesters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s);

b) esters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ hydroxylated fatty polyacids, for instance diesters of polyglycerol comprising from 2 to 10 glycerol units and of hydroxylated fatty polyacids, in particular from 5 to 25 hydroxylated C₁₂-C₂₄ fatty acids, preferably from 6 to 15 hydroxylated C₁₆-C₂₀ fatty acids;

c) ethers of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty alcohols;

d) esters of sorbitol and/or of sorbitan and of linear or branched, preferably linear, saturated or unsaturated, preferably branched or unsaturated, better still unsaturated, C₆-C₃₀, preferably C₈-C₂₄, better still C₁₀-C₂₀ fatty acid(s),

e) esters of sucrose and of linear or branched, saturated or unsaturated, preferably branched or unsaturated, better still unsaturated, C₆-C₃₀, preferably C₈-C₂₄, better still C₁₀-C₂₀ fatty acid,

f) esters of methylglucose and of linear or branched, saturated or unsaturated, preferably branched or unsaturated, better still unsaturated, C₆-C₃₀, preferably C₈-C₂₄, better still C₁₀-C₂₀ fatty acid,

g) and mixtures thereof.

The surfactant(s) with a melting point of less than or equal to 25° C. are preferentially chosen from esters of (poly)glycerol comprising from 1 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s), and mixtures thereof. More preferably, the surfactant(s) with a melting point of less than or equal to 25° C. are chosen from monoesters of glycerol and of linear or branched, saturated or unsaturated, preferably branched or unsaturated, better still unsaturated, C₆-C₃₀, preferably C₈-C₂₄, better still C₁₀-C₂₀ fatty acid(s). Particularly preferably, the surfactant with a melting point of less than or equal to 25° C. is diglyceryl monooleate.

Preferably, the total content of surfactant(s) with a melting point of less than or equal to 25° C. ranges from 0.5% to 15% by weight, preferably from 1% to 10% by weight, relative to the total weight of the composition.

Advantageously, the weight ratio between the total content of the fatty substances (i.e. of the fatty substance(s) with a melting point of less than or equal to 25° C. and of the fatty substances with a melting point of greater than 25° C.) and the total content of the surfactant(s) with a melting point of less than or equal to 25° C. is greater than or equal to 10, preferably greater than or equal to 15.

Additives

The composition according to the present invention may also optionally comprise one or more additives, different from the compounds of the invention and among which mention may be made of nonionic, cationic, anionic, amphoteric or zwitterionic surfactants other than those of the invention, and mixtures thereof, cationic, anionic, nonionic or amphoteric polymers, or mixtures thereof, antidandruff agents, anti-seborrhoea agents, vitamins and provitamins including panthenol, sunscreens, mineral or organic pigments, sequestrants, plasticizers, solubilizers, acidifying agents, mineral or organic thickeners, notably polymeric thickeners, opacifiers, nacreous agents, antioxidants, hydroxy acids, fragrances, preserving agents and pigments.

Needless to say, a person skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of the composition.

The composition according to the invention is in the form of a foam.

Preferably, the composition according to the invention is in foam form when it is applied to the keratin materials.

In this embodiment, the generation of the foam may be performed at the time of use.

In a preferred embodiment, the anhydrous composition according to the invention is obtained by placing the liquid and solid fatty substances and the liquid surfactant(s) in contact for a few minutes at a temperature in the region of 60-70° C. After cooling, a foam is obtained by mechanical shearing, for instance with a whisk or an electric blender, preferably performed just before application.

The mechanical shearing may be performed with a whisk, a system for expansion by air injection, or a pump bottle equipped with a push button which generates a foam.

The generated foam can be characterized by measuring its overrun, i.e. the volume percentage of air incorporated into the foam oil according to the following equation:

$$Overrun = \frac{{V}_{air}}{{V}_{air} + {V}_{oil}}$$

where Vair and Voil are the volumes of air and the oil phase in the foam, respectively.

The overrun can be determined at room temperature (25° C.) using the method described by M. Brun, M. Delample, E. Harte, S. Lecomte, F. Leal-Calderon, Stabilization of air bubbles in oil by surfactant crystals: A route to produce air-in-oil foams and air-in-oil-in-water emulsions, Food Res. Int. 67 (2015) 366-375.

Advantageously, the overrun of the foam compositions of the present invention ranges from 25% to 80%, preferably from 30% to 60%.

In a particular embodiment, the composition according to the invention is used for the cleansing and/or cosmetic care of the skin, in particular a composition for treating dry skin.

In another embodiment of the invention, the composition according to the invention is used for the cleansing and/or cosmetic care of the hair and/or the scalp, in particular as a treatment before shampooing, or as shampoo or as hair conditioner.

The invention also relates to a cosmetic process for treating keratin materials, in particular human keratin materials such as the hair and the skin, comprising the application to said keratin materials of an anhydrous cosmetic composition as defined previously.

The composition according to the invention may be applied to wet or dry keratin materials that have optionally been washed beforehand, for example with a shampoo.

On conclusion of the cosmetic treatment, the keratin materials are optionally rinsed with water, optionally washed and then rinsed with water, before being dried or left to dry.

Preferably, the step of applying the composition according to the invention is followed by a step of rinsing the keratin materials, preferably with water.

The composition according to the present invention is applied for a leave-on time that may range from 1 minute to 2 hours, preferably from 2 to 10 minutes.

The present invention also relates to the use of an anhydrous cosmetic composition as defined previously for washing and/or conditioning keratin materials, in particular human keratin materials such as the hair or the skin, and preferably the hair.

The examples that follow serve to illustrate the invention without, however, being limiting in nature.

### EXAMPLES

In the examples that follow, all the amounts are given as mass percentages of active material (AM) relative to the total weight of the composition.

### Example 1

a) Test Compositions

Composition B1 according to the present invention and comparative composition A1 were prepared using the ingredients whose contents are indicated in the table below:

TABLE 1

| | A1 | B1 |
|---|---|---|
| | (Comparative) | (Invention) |
| Sunflower oil | 90 | 88 |
| Behenyl alcohol | 10 | 10 |
| Diglyceryl monooleate | — | 2 |

Foam Generation Protocol:

Foams were obtained at room temperature (25° C.) by introducing 100 g of each composition in a 75 cL plastic beaker (Kenwood). Then, a whisk (Kenwood Triblade Hand Blender HDP304WH) has been used at speed 5 for 3 minutes to incorporate air bubbles in the composition and produce the oil foam. Each foam was prepared in triplicate.

The overrun was determined according to the following equation:

$$Overrun = \frac{{V}_{air}}{{V}_{air} + {V}_{oil}}$$

where Vair and Voil are the volumes of air and the oil phase in the foam, respectively, using the protocol described by Brun et al. (Stabilization of air bubbles in oil by surfactant crystals: A route to produce air-in-oil foams and air-in-oil-in-water emulsions, Food Res. Int. 67 (2015)). Straight after the foaming process, the foams were introduced in a 25 mL measuring cylinder corresponding to Vair+Voil. Then, the foam was heated to 65° C. in a water bath. At 65° C., the foam was completely destabilized due to the melting of the crystalline particles stabilizing the foams leading to the determination of the final volume Voil. The overrun has been measured for three different foams in order to calculate the average and the standard deviation.
The overrun of the foam generated from the composition B1 is 38+/−5, which is significantly higher than the overrun of the foam generated from comparative composition A1 (22+/−2).
Then, the foam was used just after generation, according to the procedure described below.
b) Procedure

Compositions A1 and B1 thus obtained were applied to 2.7 g locks of dry natural hair, at a rate of 1 g of composition per lock of hair.

All the locks were first subjected to standard shampooing (1 g of shampoo for a 2.7 g lock) and then left to dry naturally. Next, 1 g of foam to be applied is weighed out in a watch glass. The lock is then rolled around the fingers and the foam to be applied is taken up with the rolled-up lock. The foam is then spread over the entire length of the lock. The lock is massaged over the entire length by passing the fingers up and down five times. The lock is then combed five times on a clean surface with a large-toothed comb.

The rinseability of the compositions was evaluated by an expert according to the following protocol.

After a leave-on time of 5 minutes at room temperature, the locks were placed a first time vertically under a tap of water, the temperature of which was 35° C. and the flow rate of which was 2 litres/minute. After 5 seconds, the locks were drained lightly between the thumb and index finger out of the stream of water, corresponding to a first pass. The locks of hair were then placed a second time vertically under a tap of water, the temperature of which was 35° C. and the flow rate of which was 2 litres/minute. After 5 seconds, the locks were drained lightly between the thumb and index finger out of the stream of water, corresponding to a second pass.

This operation was repeated until a lock which did not feel greasy was obtained. The results obtained are given in the table below (mean for three locks).

TABLE 2

| | A1 | B1 |
|---|---|---|
| | (comparative) | (invention) |
| Number of | 13 | 5 |
| passes required | | |
| for rinsing | | |

Composition B1 according to the invention has better rinseability than the comparative composition A1. Specifically, the lock treated with the composition according to the invention requires only five passes under the water, whereas the lock treated with the comparative composition requires 13 passes under the water.

After rinsing, the locks were then placed in the open air for natural drying before evaluation by the expert. After drying, the cosmetic properties of the locks, and notably the feel, were evaluated.

c) Results

Composition B1 according to the invention gives the hair better cosmetic properties than the comparative composition.

Specifically, the lock treated with composition B1 is soft with a light coating but without a thick, greasy film, unlike the lock treated with the comparative composition A1 which remains with a greasy feel.

### Example 2

a) Test Compositions

Composition B1 according to the present invention and comparative composition A1 were prepared using the ingredients whose contents are indicated in Example 1.

b) Procedure

The foams obtained using compositions A1 and B1 (see the protocol described in example 1) were applied to 2.7 g locks of wet hair, washed beforehand with a shampoo (1 g of shampoo for a 2.7 g lock). Next, 1 g of foam to be applied is weighed out in a watch glass. The lock is then rolled around the fingers and the foam to be applied is taken up with the rolled-up lock. The foam is then spread over the entire length of the lock. The lock is massaged over the entire length by passing the fingers up and down five times. The lock is then combed five times on a clean surface with a large-toothed comb.

The rinseability of the compositions was evaluated by an expert according to the protocol described in Example 1b.

The results obtained are given in the table below (mean for three locks).

TABLE 3

| | A1 | B1 |
|---|---|---|
| | (comparative) | (invention) |
| Number of | 15 | 6 |
| passes required | | |
| for rinsing | | |

Composition B1 according to the invention has better rinseability than the comparative composition A1. Specifically, the lock treated with the composition according to the invention requires only six passes under the water, whereas the lock treated with the comparative composition requires 15 passes under the water.

After rinsing, the locks were then dried with a hairdryer before evaluation by the expert. After drying, the cosmetic properties of the locks, and notably the feel, were evaluated visually.

c) Results

Composition B1 according to the invention gives the hair better cosmetic properties than the comparative composition.

Specifically, the lock treated with composition B1 is soft with a light coating but without a thick, greasy film, unlike the lock treated with the comparative composition A1 which leaves a greasy film after drying with the hairdryer.

### Example 3

a) Test Compositions

Composition B2 according to the present invention and comparative compositions A1 and A2 were prepared using the ingredients whose contents are indicated in the table below:

TABLE 4

| | A1 | A2 | B2 |
|---|---|---|---|
| | (Comparative) | (Comparative) | (Invention) |
| Sunflower oil | 90 | 85 | 85 |
| Behenyl alcohol | 10 | 10 | 10 |
| Diglyceryl monooleate | — | — | 5 |
| (melting point less than | | | |
| 25° C.) | | | |
| Steareth-2 | — | 5 | — |
| (melting point greater | | | |
| than 25° C.) | | | |

Foam Generation Protocol:

The foams were prepared using the protocol described in example 1.

The overrun of the foams were measured according to the method described in example 1. The overrun of the foam generated from composition B1 is of 34 (+/−3) whereas the overrun of the foams generated from compositions A1 and A2 are of 22 (+/−2) and 15 (+/−4), respectively.
The foam was used just after generation, according to the procedure described below.

b) Procedure

The foams obtained using compositions A1, A2 and B2 thus obtained were applied to locks of dry hair in the same manner as in Example 1b above. The rinsing protocol described in Example 1b was then applied.

The same protocol for application to the hair was used.

The results obtained are given in the table below (mean for three locks).

TABLE 5

| | A1 | A2 | B2 |
|---|---|---|---|
| | (comparative) | (comparative) | (invention) |
| Number of | 15 | 21 | 3 |
| passes required | | | |
| for rinsing | | | |

Composition B2 according to the invention has better rinseability than the comparative compositions A1 and A2. Specifically, the lock treated with the composition according to the invention requires only three passes under the water, whereas the lock treated with comparative composition A1 requires 15 passes under the water and with comparative composition A2, 21 passes on average.

After rinsing, the locks were then dried with a hairdryer before evaluation by the expert. After drying, the cosmetic properties of the locks were evaluated visually and by touch.

c) Results

The composition according to the invention, containing a surfactant with a melting point of less than or equal to 25° C. gives better cosmetic properties than the comparative compositions not comprising any surfactant (A1) or comprising a surfactant with a melting point of greater than 25° C. (A2).

Specifically, the lock treated with composition B2 is soft with a light coating but without a thick, greasy film, unlike the locks treated with compositions A1 and A2. The lock treated with composition A2 is also very greasy, visually and to the touch.

### Example 4

a) Test Compositions

Two compositions were prepared using the ingredients whose contents are indicated in Example 1 for composition B1: the first composition (B1) was prepared following the protocol described in example 1 and was under the form of a foam whereas the second composition (B1′) was prepared by simple mixing of the ingredients and was not under the form of a foam.

Both compositions were then applied on dry hair and their rinseability was evaluated according to the procedure described in example 1.

c) Results

The results obtained regarding the rinseability of the composition are given in the table below (mean for three locks).

TABLE 6

| | B1′ not foam | B1 foam |
|---|---|---|
| | (comparative) | (invention) |
| Number of | 9 | 5 |
| passes required | | |
| for rinsing | | |

Composition B1 in the form a foam (according to the invention) has better rinseability than the comparative composition which is not in the form of a foam. Specifically, the lock treated with the foam composition B1 requires only five passes under the water to be totally rinsed off, whereas the lock treated with the comparative composition B1′ requires 9 passes under the water.

After rinsing, the locks were then placed in the open air for natural drying before evaluation by the expert. After drying, the cosmetic properties of the locks, and notably the feel, were evaluated.

Composition B1 in the form a foam (according to the invention) gives the hair better cosmetic properties than the comparative composition (not foam).

Specifically, the lock treated with the foam composition B1 is soft with a light coating but without any thick, greasy film, whereas the lock treated with the comparative composition B1′ have a greasy touch.

1. Anhydrous cosmetic composition comprising:
one or more fatty substances with a melting point of less than or equal to 25° C.,
one or more fatty substances with a melting point of greater than 25° C., and
one or more surfactants with a melting point of less than or equal to 25° C.,
said composition being in the form of a foam.

2. Composition according to claim 1, characterized in that the fatty substance(s) with a melting point of less than or equal to 25° C. are chosen from C6-C16 liquid hydrocarbons, liquid hydrocarbons comprising more than 16 carbon atoms, non-silicone oils of animal origin, oils of triglyceride type of plant or synthetic origin, fluoro oils, liquid fatty alcohols, liquid fatty acid and/or fatty alcohol esters other than triglycerides, and silicone oils, and mixtures thereof.

3. Composition according to claim 1, characterized in that the fatty substance(s) with a melting point of less than or equal to 25° C. are chosen from oils of triglyceride type of plant origin, and mixtures thereof, preferably from sunflower oil, soybean oil, olive oil, avocado oil, and mixtures thereof.

4. Composition according to claim 1, characterized in that the total content of the fatty substance(s) with a melting point of less than or equal to 25° C. is greater than or equal to 70% by weight, preferably greater than or equal to 75% by weight, more preferentially ranges from 75% to 95% by weight and better still from 80% to 90% by weight, relative to the total weight of the composition.

5. Composition according to claim 1, characterized in that the fatty substance(s) with a melting point of greater than 25° C. are chosen from solid fatty acids, solid fatty alcohols, solid esters of fatty acids and/or of fatty alcohols, waxes, ceramides, monoglycerides, diglycerides or triglycerides, and mixtures thereof, preferably from solid fatty acids, solid fatty alcohols, and mixtures thereof, and more preferentially from behenyl alcohol, behenic acid, and mixtures thereof.

6. Composition according to claim 1, characterized in that the total content of the fatty substance(s) with a melting point of greater than 25° C. ranges from 1% to 20% by weight, preferably from 5% to 15% by weight, relative to the total weight of the composition.

7. Composition according to claim 1, characterized in that the weight ratio between the total content of the fatty substance(s) with a melting point of less than or equal to 25° C. and the total content of the fatty substance(s) with a melting point of greater than 25° C. is greater than or equal to 3.5, preferably greater than or equal to 5.

8. Composition according to claim 1, characterized in that the total content of the fatty substance(s) with a melting point of less than or equal to 25° C. and of the fatty substance(s) with a melting point of greater than 25° C. is greater than or equal to 75% by weight, preferably greater than or equal to 80% by weight, preferentially greater than or equal to 85% by weight, relative to the total weight of the composition.

9. Composition according to claim 1, characterized in that the surfactant(s) with a melting point of less than or equal to 25° C. are chosen from nonionic surfactants, and mixtures thereof.

10. Composition according to claim 1, characterized in that the surfactant(s) with a melting point of less than or equal to 25° C. have an HLB value ranging from 1 to 9 and preferably from 1 to 7.

11. Composition according to claim 1, characterized in that the surfactant(s) with a melting point of less than or equal to 25° C. are chosen from esters of (poly)glycerol comprising from 1 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s); esters of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ hydroxylated fatty polyacids; ethers of polyglycerol comprising from 2 to 10 glycerol units and of linear or branched, saturated or unsaturated, preferably branched or unsaturated, better still unsaturated, C₆-C₃₀, preferably C₈-C₂₄, better still C₁₀-C₂₀ fatty alcohol; esters of sorbitol and/or of sorbitan and of linear or branched, saturated or unsaturated, preferably branched or unsaturated, better still unsaturated, C₆-C₃₀, preferably C₈-C₂₄, better still C₁₀-C₂₀ fatty acid(s); esters of sucrose and of linear or branched, saturated or unsaturated, preferably branched or unsaturated, better still unsaturated, C₆-C₃₀, preferably C₈-C₂₄, better still C₁₀-C₂₀ fatty acids; esters of methylglucose and of linear or branched, saturated or unsaturated, preferably branched or unsaturated, better still unsaturated, C₆-C₃₀, preferably C₈-C₂₄, better still C₁₀-C₂₀ fatty acid; and mixtures thereof, preferably from esters of (poly)glycerol comprising from 1 to 10 glycerol units and of linear or branched, saturated or unsaturated, preferably branched or unsaturated, better still unsaturated, C₆-C₃₀, preferably C₈-C₂₄, better still C₁₀-C₂₀ fatty acid(s), and mixtures thereof.

12. Composition according to claim 1, characterized in that the content of the surfactant(s) with a melting point of less than or equal to 25° C. ranges from 0.5% to 15% by weight, preferably from 1% to 10% by weight, relative to the total weight of the composition.

13. Composition according to claim 1, characterized in that the weight ratio between the total content of the fatty substance(s) and the total content of the surfactant(s) with a melting point of less than or equal to 25° C. is greater than or equal to 10, preferably greater than or equal to 15.

14. Cosmetic process for treating keratin materials, in particular human keratin materials such as the hair and the skin, comprising the application to said keratin materials of a composition as defined in claim 1.

15. Use of the cosmetic composition as defined according to claim 1, for washing and/or conditioning keratin materials, notably human keratin materials such as the hair and the skin.
